(19) United States
(12) Patent Application Publication
(10) Pub. No.: US 2012/0029041 A1
(43) Pub. Date: Feb. 2, 2012

(54) 2-ARYL IMIDAZOLINE DERIVATIVES

(75) Inventors: Yuji Haga, Ibaraki (JP); Minoru Moriya, Ibaraki (JP); Toshiyuki Takahashi, Ibaraki (JP)

(73) Assignee: MSD K.K., Chiyoda-ku (JP)

(21) Appl. No.: 13/259,630

(22) PCT Filed: Apr. 21, 2010
(86) PCT No.: PCT/JP2010/057533

§ 371 Date: Oct. 18, 2011

(30) Foreign Application Priority Data
Apr. 22, 2009 (JP) 2009-104436

(51) Int. Cl. A61K 31/4164; A61P 3/04; C07D 233/60

(52) U.S. Cl. 514/399; 548/341.1

(57) ABSTRACT

2-Aryl imidazoline derivatives are provided that are useful as anti-obesity agents or the like. A compound represented by Formula (I) or a pharmaceutically acceptable salt thereof: wherein R¹ and R² each independently represent a hydrogen atom, halogen, C₁₋₆alkyl, or the like; R^{3a}, R^{3b}, R^{4a} and R^{4b} each independently represent a hydrogen atom, C₁₋₆alkyl, or haloC₁₋₆alkyl; and Ar represents aryl, wherein the aryl may be substituted with 1 to 3 substituents such as halogen, cyano, C₁₋₆alkyl, haloC₁₋₆alkyl, C₁₋₆alkyloxy, haloC₁₋₆alkyloxy, hydroxy, amino, monoC₁₋₆alkylamino, diC₁₋₆alkylamino, and C₁₋₆alkyloxy-carbonyl.

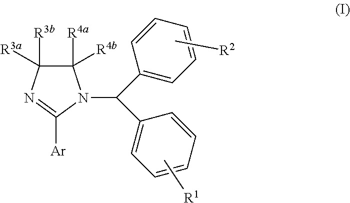

### TECHNICAL FIELD

The present invention relates to 2-aryl imidazoline derivatives useful as medicaments. The compounds act as human QRFP receptor (GPR103) antagonists, and are useful as a preventive or remedy for obesity and other diseases.

### BACKGROUND ART

QRFP43, a peptide of 43 amino acids, has been reported as an endogenous ligand of QRFP43 receptor (GPR103) through bioinformatics and reverse pharmacology (2003), and it was first isolated from rat brains in 2006 (see, for example, Non-Patent Document 1). There are also reports that, for example, 26RFa, a close relative of QRFP43, binds to QRFP43 receptor, and has activity similar to that of QRFP43 (see, for example, Patent Documents 1 and 2). QRFP43 is expressed at high level in the central nervous system, particularly in the hypothalamus, and has a diversity of functions in the body. Specifically, QRFP43 acts as a centrally-acting appetite promoter, and promotes prominent fat accumulation through secretion of various hormones or actions of the nervous system. It is known that intracerebroventricular successive administration of QRFP43 induces obesity and insulin resistance based on these actions. QRFP43 is also involved in hormone secretion such as in the hypothalamus and pituitary gland.

The functions of QRFP43 or 26RFa are expressed upon binding to the QRFP43 receptor (GPR103) present in the central or peripheral nervous system. It would therefore be possible to inhibit the functional expression of QRFP43 or 26RFa by inhibiting the binding of QRFP43 or 26RFa to the QRFP43 receptor (GPR103).

Bis-(4-fluorophenyl)-methyl]-4,5-dihydro-2-methylimidazole disclosed in, for example, Patent Document 3 is a compound relating to the 2-aryl imidazoline derivatives of the present invention. However, in this compound, the moiety corresponding to the Ar of the compounds of the present invention is a methyl, and the compound differs from the present invention.

Non-Patent Document 1: The Proceedings of the National Academy of Sciences, Vol. 103, pp. 7438-7443, (2006)

Patent Document 1: WO01/16316

Patent Document 2: WO05/65702

Patent Document 3: U.S. Pat. No. 3,823,155A

### DISCLOSURE OF THE INVENTION

### Problems that the Invention is to Solve

Because the inhibition of the QRFP43 or 26RFa binding to the QRFP43 receptor (GPR103) can inhibit the functional expression of QRFP43 or 26RFa, a substance antagonistic to the QRFP43 binding to the QRFP43 receptor (GPR103) is expected to be useful in the prevention or treatment of various diseases involving QRFP43 or 26RFa, including, for example, cardiovascular disease such as hypertension, arterial sclerosis, renal disease, heart disease, and angiospasm; bulimia; and metabolic disease such as obesity, diabetes mellitus, abnormal hormone secretion, hypercholesteremia, hyperlipidemia, gout, and fatty liver. Such a substance can therefore be provided as a preventive or remedy for, for example, pain, abnormal circadian rhythms, atherosclerosis, obesity-related gastroesophageal reflux, obesity-hypoventilation syndrome (Pickwickian syndrome), hypertriglyceridemia, low HDL cholesteremia, cardiovascular disease (for example, coronary artery heart disease (CHD), cerebrovascular disease, stroke, peripheral vascular disease, and sudden death), pain, osteoporosis-related disease, lower back pain, and anesthetic hypersensitivity.

It is accordingly an object of the present invention to provide an antagonist for QRFP43 receptor (GPR103), which is useful as a preventive or remedy for diseases such as above.

### Means for Solving the Problems

The inventors of the present invention have found that a specific compound having a diphenylmethyl substituent at position 1 of imidazoline, and an aryl substituent at position 2 acts as a human QRFP receptor (GPR103) antagonist. The present invention was completed based on this finding.

Specifically, the present invention provides:

(1) A compound represented by Formula (I) or a pharmaceutically acceptable salt thereof:

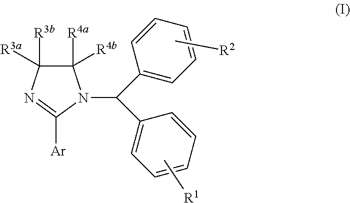

wherein R¹ and R² each independently represent a hydrogen atom, halogen, C₁₋₆alkyl, haloC₁₋₆alkyl, C₁₋₆alkyloxy, or haloC₁₋₆alkyloxy;

R^{3a}, R^{3b}, R^{4a}, and R^{4b} each in dependently represent a hydrogen atom, C₁₋₆alkyl, or haloC₁₋₆alkyl; and

Ar represents aryl, wherein the aryl may be substituted with 1 to 3 substituents selected from a group consisting of halogen, cyano, C₁₋₆alkyl, haloC₁₋₆alkyl, C₁₋₆alkyloxy, haloC₁₋₆alkyloxy, hydroxy, amino, monoC₁₋₆alkylamino, diC₁₋₆alkylamino, C₁₋₆alkyloxycarbonyl, C₁₋₆alkylcarbonylamino, C₁₋₆alkylcarbonyl(C₁₋₆alkyl)amino, C₁₋₆alkyloxycarbonylamino, C₁₋₆alkyloxycarbonyl(C₁₋₆alkyl)amino, C₁₋₆alkylsulfanyl, C₁₋₆alkylcarbonyl, and C₁₋₆alkylcarbonyloxy.

(2) A pharmaceutical composition which contains the compound of (1) or the pharmaceutically acceptable salt thereof, and a pharmaceutically acceptable carrier, and

(3) A preventive or remedy for obesity, which contains the compound of (1) or the pharmaceutically acceptable salt thereof as an active ingredient.

### BEST MODE FOR CARRYING OUT THE INVENTION

The present invention is described below in more detail.

Examples of the “halogen” include fluoro, chloro, bromo, and iodo.

The “C₁₋₆alkyl” includes straight-chain alkyls having 1 to 6 carbon atoms, and branched alkyls having 3 to 6 carbon atoms. Specific examples include methyl, ethyl, n-propyl, isopropyl, n-butyl, isobutyl, sec-butyl, tert-butyl, n-pentyl, isopentyl, neopentyl, tert-amyl, 2-propyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, and 1-ethyl-1-methylpropyl.

The “haloC₁₋₆alkyl” includes C₁₋₆alkyls in which some of or all of the hydrogen atoms are substituted with halogens. Examples include fluoromethyl, difluoromethyl, trifluoromethyl, 2-fluoroethyl, and 1,2-difluoroethyl.

The “C₁₋₆alkyloxy” includes groups with the C₁₋₆alkyl attached to an oxygen atom. Specific examples include methoxy, ethoxy, n-propyloxy, isopropyloxy, n-butoxy, isobutoxy, tert-butoxy, and n-pentyloxy.

The “haloC₁₋₆alkyloxy” includes groups with the haloC₁₋₆alkyl attached to an oxygen atom. Specific examples include fluoromethoxy, chloromethoxy, difluoromethoxy, dichloromethoxy, trifluoromethoxy, trichloromethoxy, 2-fluoroethoxy, and 1,2-difluoroethoxy.

The “monoC₁₋₆alkylamino” is a group in which one of the hydrogen atoms of the amino (—NH₂) is substituted with a C₁₋₆alkyl group. Specific examples include methylamino, ethylamino, n-propylamino, isopropylamino, n-butylamino, sec-butylamino, and tert-butylamino.

The “diC₁₋₆alkylamino” is a group in which the two hydrogen atoms of the amino are substituted with C₁₋₆alkyls. Specific examples include dimethylamino, diethylamino, ethylmethylamino, di(n-propyl)amino, methyl(n-propyl)amino, and diisopropylamino.

The “C₁₋₆alkyloxycarbonyl” is a group with the C₁₋₆alkyloxy attached to a carbonyl (—CO—), and includes alkyloxycarbonyls having 1 to 6 carbon atoms. Specific examples include methoxycarbonyl, ethoxycarbonyl, n-propyloxycarbonyl, isopropyloxycarbonyl, n-butoxycarbonyl, isobutoxycarbonyl, tert-butoxycarbonyl, and n-pentyloxycarbonyl.

The “C₁₋₆alkylcarbonyl” is a group with the C₁₋₆alkyl attached to a carbonyl, and includes alkylcarbonyls having 1 to 6 carbon atoms. Specific examples include acetyl, propionyl, butyryl, isobutyryl, valeryl, isovaleryl, and pivaloyl.

The “C₁₋₆alkylcarbonylamino” is a group in which one of the hydrogen atoms of the amino group is substituted with a C₁₋₆alkylcarbonyl. Specific examples include acetylamino, propionylamino, isobutyrylamino, valerylamino, isovalerylamino, and pivaloylamino.

The “C₁₋₆alkylcarbonyl(C₁₋₆alkyl)amino” is a group in which the hydrogen atom on the nitrogen atom of the monoC₁₋₆alkylamino is substituted with a C₁₋₆alkylcarbonyl. Examples include acetyl(methyl)amino, propionyl(methyl)amino, and butyryl(methyl)amino.

The “C₁₋₆alkyloxycarbonylamino” is a group in which one of the hydrogen atoms of the amino is substituted with a C₁₋₆alkyloxycarbonyl, and includes alkyloxycarbonylaminos having 1 to 6 carbon atoms. Specific examples include methoxycarbonylamino, ethoxycarbonylamino, n-propyloxycarbonylamino, isopropyloxycarbonylamino, n-butoxycarbonylamino, isobutoxycarbonylamino, tert-butoxycarbonylamino, and n-pentyloxycarbonylamino.

The “C₁₋₆alkyloxycarbonyl(C₁₋₆alkyl)amino” is a group in which a C₁₋₆alkyloxycarbonyl is attached in place of the hydrogen atom on the nitrogen atom of the monoC₁₋₆alkylamino. Specific examples include methoxycarbonyl(methyl)amino, ethoxycarbonyl(methyl)amino, and n-propyloxycarbonyl(methyl)amino.

The “C₁₋₆alkylcarbonyloxy” is a group with the C₁₋₆alkylcarbonyl attached to an oxygen atom. Specific examples include acetoxy, propionyloxy, valeryloxy, isovaleryloxy, and pivaloyloxy.

The “C₁₋₆alkylsulfanyl”, also referred to as alkylthio, is a group with the C₁₋₆alkyl attached to a sulfur atom. Specific examples include methylsulfanyl, ethylsulfanyl, n-propylsulfanyl, isopropylsulfanyl, and n-butylsulfanyl.

Examples of the “aryl” include phenyl and naphthyl.

The “pharmaceutically acceptable salt” of a derivative represented by Formula (I) includes pharmaceutically acceptable salts commonly used. Examples include an acid addition salt formed at the amine moiety of a compound of Formula (I), an acid addition salt formed at the nitrogen-containing heterocyclic ring, and, when a compound of Formula (I) has an acidic substituent, a base addition salt formed at such a group.

Examples of the acid addition salt include: inorganic acid salts such as hydrochloride, sulfate, nitrate, phosphate, and perchlorate; organic acid salts such as maleate, fumarate, tartrate, citrate, ascorbate, and trifluoroacetate; and sulfonates such as methanesulfonate, isothiocyanate, benzenesulfonate, and p-toluenesulfonate.

Examples of the base addition salt include: alkali metal salts such as sodium salt and potassium salt; alkali-earth metal salts such as calcium salt and magnesium salt; and organic amine salts such as ammonium salt, trimethylamine salt, triethylamine salt, dicyclohexylamine salt, ethanolamine salt, diethanolamine salt, triethanolamine salt, procaine salt, and N,N′-dibenzylethylenediamine salt.

The following discloses derivatives of the present invention in more detail with reference to specific examples of various symbols used in Formula (I).

R¹ and R² each independently represent a hydrogen atom, halogen, C₁₋₆alkyl, haloC₁₋₆alkyl, C₁₋₆alkyloxy, or haloC₁₋₆alkyloxy.

Specifically, R¹ and R² each independently represent, for example, a hydrogen atom; halogen such as fluoro, chloro, bromo, and iodo; C₁₋₆alkyl such as methyl, ethyl, n-propyl, isopropyl, n-butyl, and t-butyl; haloC₁₋₆alkyl such as chloromethyl, trichloromethyl, fluoromethyl, trifluoromethyl, chloroethyl, and fluoroethyl; C₁₋₆alkyloxy such as methoxy, ethoxy, n-propyloxy, and isopropyloxy; and haloC₁₋₆alkyloxy such as chloromethoxy, trichloromethoxy, fluoromethoxy, trifluoromethoxy, fluoroethoxy, and fluoropropyloxy. Preferably, R¹ and R² are each independently selected from the group consisting of a hydrogen atom, halogen, and C₁₋₆alkyl.

R^{3a}, R^{3b}, R^{4a}, and R^{4b} each independently represent a hydrogen atom, C₁₋₆alkyl, or haloC₁₋₆alkyl.

Specifically, R^{3a}, R^{3b}, R^{4a}, and R^{4b} each independently represent, for example, a hydrogen atom; C₁₋₆alkyl such as methyl, ethyl, n-propyl, isopropyl, n-butyl, and t-butyl; and haloC₁₋₆alkyl such as chloromethyl, trichloromethyl, fluoromethyl, trifluoromethyl, chloroethyl, and fluoroethyl. Preferably, a hydrogen atom or C₁₋₆alkyl is recommended.

Ar represents aryl, wherein the aryl may be substituted with 1 to 3 substituents selected from a group consisting of halogen, cyano, C₁₋₆alkyl, haloC₁₋₆alkyl, C₁₋₆alkyloxy, haloC₁₋₆alkyloxy, hydroxy, amino, monoC₁₋₆alkylamino, diC₁₋₆alkylamino, C₁₋₆alkyloxycarbonyl, C₁₋₆alkylcarbonylamino, C₁₋₆alkylcarbonyl(C₁₋₆alkyl)amino, C₁₋₆alkyloxycarbonylamino, C₁₋₆alkyloxycarbonyl(C₁₋₆alkyl)amino, C₁₋₆alkylsulfanyl, C₁₋₆alkylcarbonyl, and C₁₋₆alkylcarbonyloxy.

Examples of the aryl for Ar include phenyl and naphthyl. Preferably, phenyl is recommended.

Preferably, Ar is either unsubstituted or optionally substituted with a substituent, for which halogens such as fluoro and chloro; cyanos; C₁₋₆alkyls such as methyl, ethyl, and isopropyl; haloC₁₋₆alkyls such as fluoromethyl and trifluoromethyl; C₁₋₆alkyloxys such as methoxy, ethoxy, and isopropyloxy; haloC₁₋₆alkyloxys such as fluoromethoxy and trifluoromethoxy; C₁₋₆alkylcarbonylaminos such as methylcarbonylamino and ethylcarbonylamino; C₁₋₆alkylsulfanyls such as methylsulfanyl and ethylsulfanyl; and C₁₋₆alkylcarbonyls such as methylcarbonyl and ethylcarbonyl are recommended. More preferably, halogen, cyano, C₁₋₆alkyl, haloC₁₋₆alkyl, C₁₋₆alkyloxy, haloC₁₋₆alkyloxy, C₁₋₆alkylcarbonylamino, and C₁₋₆alkylsulfanyl are recommended.

Specific examples of Ar include phenyl, 2-chlorophenyl, 3-chlorophenyl, 4-chlorophenyl, 4-fluorophenyl, 4-methylphenyl, 4-methoxyphenyl, 4-ethoxyphenyl, 4-trifluoromethylphenyl, 4-dimethylaminophenyl, 4-acetylaminophenyl, 4-trifluoromethoxyphenyl, 4-cyanophenyl, 4-methylthiophenyl, and 4-biphenyl. Preferably, for example, phenyl, 4-chlorophenyl, 4-methylphenyl, 4-methoxyphenyl, 4-ethoxyphenyl, 4-cyanophenyl, and 4-methylthiophenyl are recommended.

A preferred embodiment of the present invention is, for example, a compound of Formula (1) or a pharmaceutically acceptable salt thereof in which:

R¹ and R² are each independently a hydrogen atom, halogen, or C₁₋₆alkyl;

R^{3a}, R^{3b}, R^{4a}, and R^{4b} are each independently a hydrogen atom or C₁₋₆alkyl; and

Ar is phenyl or naphthyl, wherein the phenyl or naphthyl may be substituted with a substituent selected from the group consisting of halogen, cyano, C₁₋₆alkyl, haloC₁₋₆alkyl, C₁₋₆alkyloxy, haloC₁₋₆alkyloxy, C₁₋₆alkylcarbonylamino, and C₁₋₆alkylsulfanyl.

A particularly preferred embodiment of the present invention is, for example, a compound of Formula (1) or a pharmaceutically acceptable salt thereof in which:

R¹ and R² are each independently a hydrogen atom, halogen, or C₁₋₆alkyl;

R^{3a}, R^{3b}, R^{4a}, and R^{4b} are each independently a hydrogen atom or C₁₋₆alkyl; and

Ar is phenyl, 4-chlorophenyl, 4-methylphenyl, 4-methoxyphenyl, 4-ethoxyphenyl, 4-cyanophenyl, or 4-methylthiophenyl.

Specific examples of compounds of the present invention include the following.

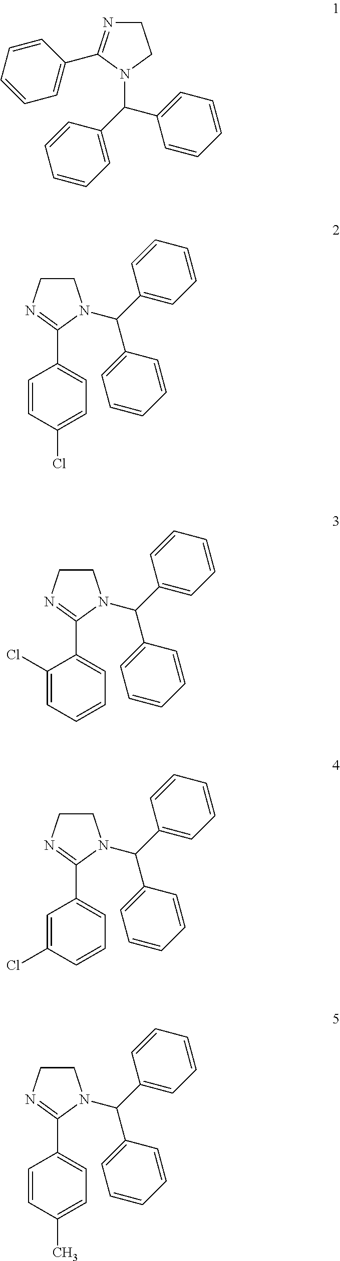

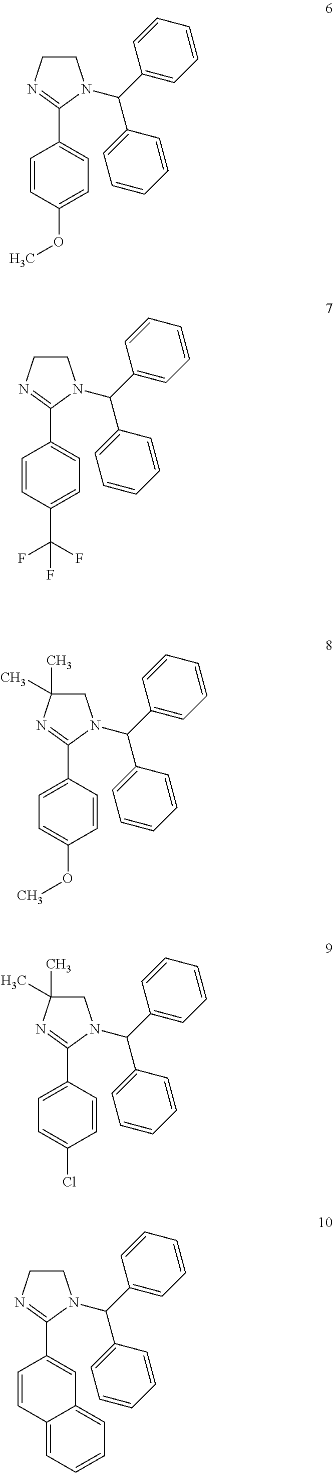

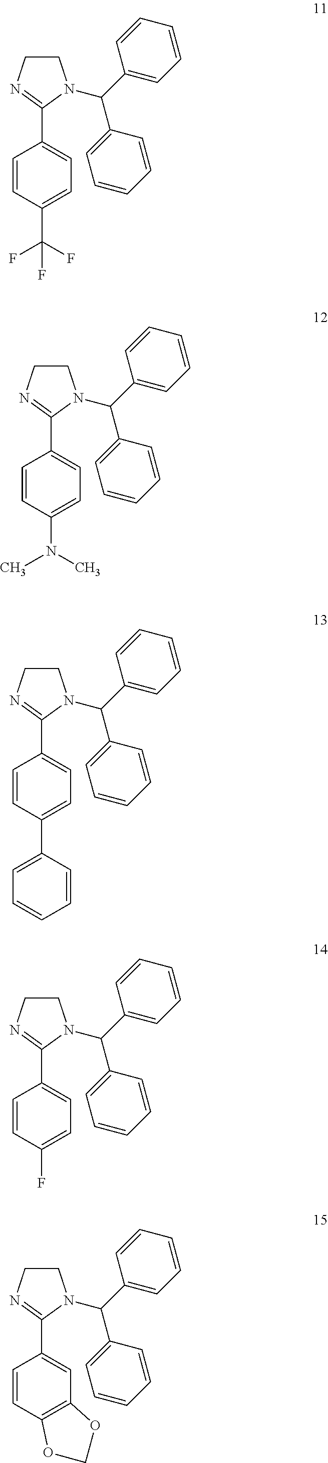

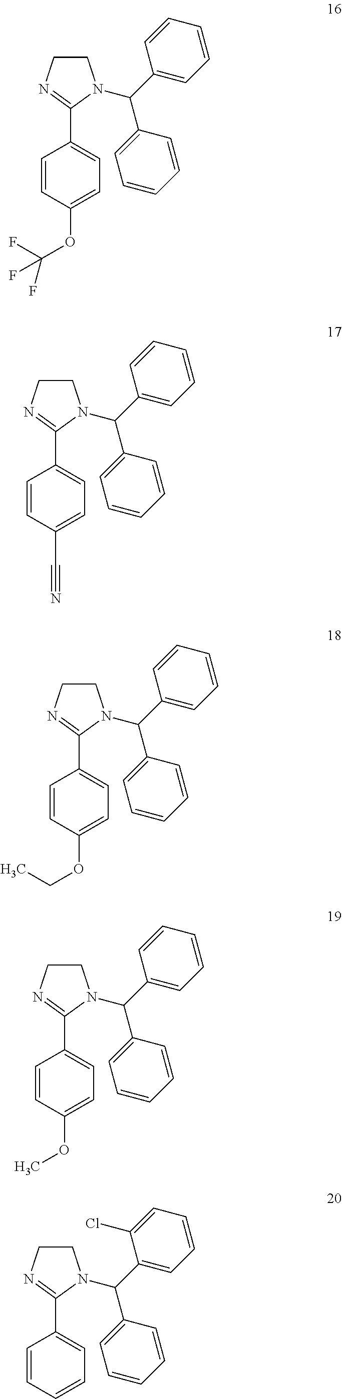

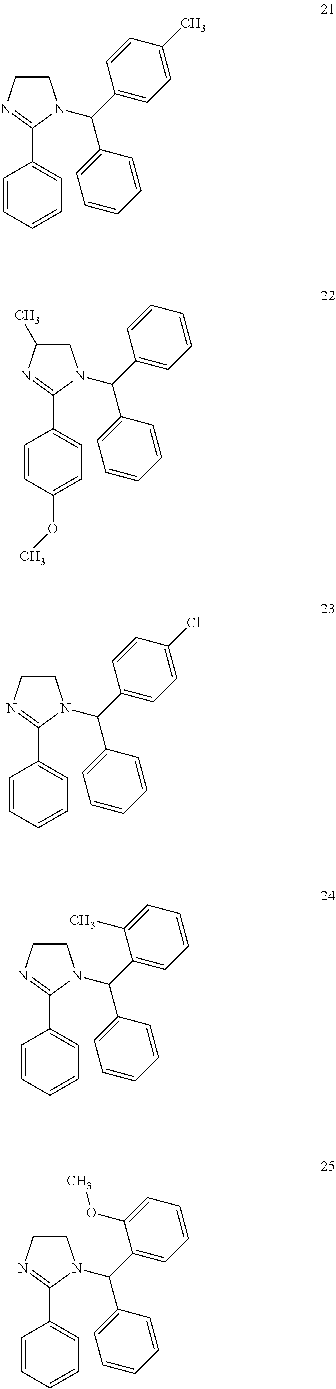

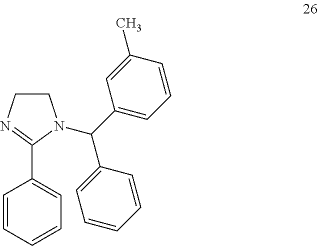

### Methods for Preparing Compounds of Formula (I)

Compounds of Formula (I) can be synthesized according to the methods described below, or by methods analogous thereto.

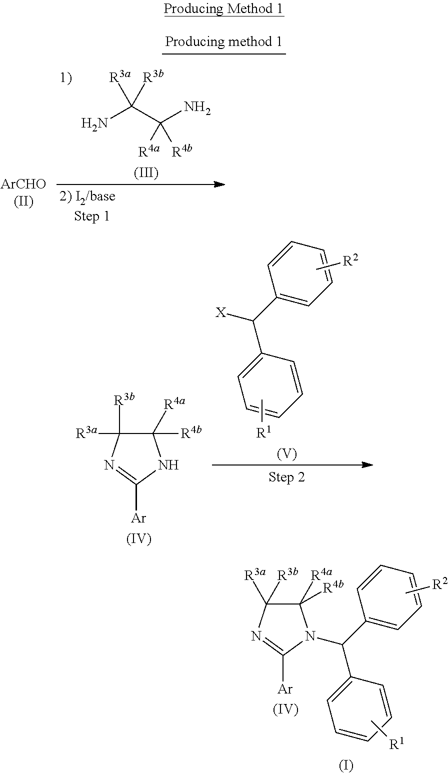

In the formulae, X represents halogen such as chloro and bromo. The other symbols are as defined above.

### Step 1

The compound of Formula (IV) can be synthesized according to the method described in Tetrahedron, 2007, Vol. 63, p. 1474, or by methods analogous thereto. Specifically, the compound of Formula (II) is reacted with the compound of Formula (III) in an organic solvent, and iodine and a base are added to the resulting reaction mixture to give the compound of Formula (IV) after further reaction.

The amount of the compound of Formula (III) used is, for example, 0.5 to 5 moles per mole of the compound of Formula (II), preferably 1 to 2 moles.

Examples of the organic solvent include t-butanol.

The reaction temperature is, for example, from room temperature to about 50° C. Iodine and a base are added to the reaction mixture after 30 to 60 minutes of reaction.

Examples of the base include potassium carbonate, and sodium carbonate.

The amount of iodine used is, for example, 0.5 to 5 moles per mole of the compound of Formula (II), preferably 1 to 1.5 moles. The amount of base used is, for example, 1 to 10 moles per mole of the compound of Formula (II), preferably 2 to 3 moles.

The reaction temperature is, for example, 20 to 150° C. Preferably, 50 to 80° C. is recommended. The reaction generally completes in 2 to 24 hours.

The reaction may be effected at 140° C. using a microwave synthesizer.

Examples of the compound of Formula (II) include benzaldehyde, 2-chlorobenzaldehyde, 3-chlorobenzaldehyde, 4-chlorobenzaldehyde, 4-fluorobenzaldehyde, anisaldehyde, 4-methylbenzaldehyde, 4-trifluoromethylbenzaldehyde, 4-dimethylaminobenzaldehyde, 4-acetamidebenzaldehyde, 4-phenylbenzaldehyde, 4-cyanobenzaldehyde, piperonal, and 2-naphthylaldehyde. Examples of the compound of Formula (III) include ethylenediamine, 2,2-dimethylethylenediamine, 1,2-propanediamine, and 2,3-butanediamine.

### Step 2

The compound of Formula (IV) is reacted with the compound of Formula (V) in an organic solvent in the presence of a base to obtain the compound of Formula (I).

The compound of Formula (V) is used in an amount of, for example, 0.8 to 10 moles per mole of the compound of Formula (IV), preferably 1 to 2 moles.

Examples of the base include sodium hydride, potassium carbonate, sodium carbonate, cesium carbonate, and diisopropylethylamine.

The amount of base used is, for example, 1 to 10 moles per mole of the compound of Formula (IV), preferably 2 to 4 moles.

Examples of the organic solvent include tetrahydrofuran (hereinafter, “THF”), 1,4-dioxane (hereinafter, “dioxane”), N,N-dimethylformamide (hereinafter, “DMF”), and N,N-dimethylacetamide (hereinafter, “DMA”).

The reaction temperature is, for example, 0 to 50° C. Preferably, 0 to 30° C. is recommended. The reaction generally completes in 3 to 24 hours.

Examples of the base include sodium hydride. The amount of base used is, for example, 1 to 5 moles per mole of the compound of Formula (IV), preferably 1 to 2 moles.

The reaction may be effected at 160° C. using a microwave synthesizer.

Examples of the compound of Formula (V) include chlorodiphenylmethane, bromodiphenylmethane, and chloro(2-chlorophenyl)phenylmethane.

In the foregoing producing methods, when the reactants of the reaction include groups, such as amino, hydroxy, carboxyl, oxo, and carbonyl, not involved in the reaction, the reactions of the producing method may be performed after appropriately protecting such an amino, hydroxy, carboxyl, oxo, or carbonyl group with an amino protective group, a hydroxy protective group, a carboxyl protective group, or an oxo or carbonyl protective group, which can be removed after the reaction.

Introduction and removal of the protective group, though it depends on the type of the protective group and the stability and other properties of the target compound, can be performed, for example, by solvolysis using an acid or a base, according to the method described in Protective Groups in Organic Synthesis, T. W. Greene, John Wiley & Sons, 1981, or methods analogous thereto; specifically, by methods employing, for example, 0.01 moles to a large excess of an acid, preferably such as trifluoroacetic acid, formic acid, or hydrochloric acid, or an equimolar amount to a large excess of a base, preferably such as potassium hydroxide or calcium hydroxide, or by methods employing chemical reduction using compounds such as a metal hydride complex, or catalytic reduction using catalysts such as palladium-carbon catalyst, and Raney nickel catalyst.

The protective group of the amino is not particularly limited, as long as it functions as intended. Examples include: aralkyls such as benzyl, p-methoxybenzyl, and trityl; lower alkanoyls such as acetyl and pivaloyl; benzoyl; lower alkyloxycarbonyls such as methoxycarbonyl, ethoxycarbonyl, and tert-butoxycarbonyl; alkyloxycarbonyls such as benzyloxycarbonyl; lower alkylsilyls such as trimethylsilyl, and tert-butyldimethylsilyl; tetrahydropyranyl; trimethylsilylethoxymethyl; lower alkylsulfonyls such as methylsulfonyl, and ethylsulfonyl; and arylsulfonyls such as benzenesulfonyl, and toluenesulfonyl. Particularly preferable examples include acetyl, benzoyl, tert-butoxycarbonyl, trimethylsilylethoxymethyl, and methylsulfonyl.

The protective group of the hydroxy is not particularly limited, as long as it functions as intended. Examples include: lower alkyls such as methyl, ethyl, and tert-butyl; lower alkylsilyls such as trimethylsilyl, and tert-butyldimethylsilyl; lower alkyloxymethyls such as methoxymethyl, and 2-methoxyethoxymethyl; tetrahydropyranyl; trimethylsilylethoxymethyl; aralkyls such as benzyl, p-methoxybenzyl, and 2,3-dimethoxybenzyl; and acyls such as acetyl. Particularly preferable examples include methyl, methoxymethyl, tetrahydropyranyl, trityl, trimethylsilylethoxymethyl, tert-butyldimethylsilyl, and acetyl.

The protective group of the carboxyl is not particularly limited, as long as it functions as intended. Examples include: lower alkyls such as methyl, ethyl, and tert-butyl; halo lower alkyls such as 2,2,2-trichloroethyl; lower alkenyls such as a 2-propenyl group; and aralkyls such as benzyl, p-methoxybenzyl, benzhydryl, and trityl. Particularly preferable examples include methyl, ethyl, tert-butyl, 2-propenyl, benzyl, p-methoxybenzyl, and benzhydryl.

The protective group of the carbonyl is not particularly limited, as long as it functions as intended. Examples include acetals and ketals such as ethylene ketal, dimethyl ketal, and S,S′-dimethyl ketal.

The compound of Formula (I) obtained as above can easily be isolated and purified using common separation means, for example, such as solvent extraction, recrystallization, column chromatography, and preparative thin-layer chromatography.

### Pharmaceutical Composition Containing a Compound of Formula (I)

A compound of Formula (I) can be orally or parenterally administered, and can be prepared into a suitable administration form expected to be useful for the prevention or treatment of, for example, cardiovascular disease such as hypertension, arterial sclerosis, renal disease, heart disease, and angiospasm; bulimia; and metabolic disease such as obesity, diabetes mellitus, abnormal hormone secretion, hypercholesteremia, hyperlipidemia, gout, and fatty liver. A compound of Formula (I) can therefore be provided as a preventive or remedy for, for example, pain, abnormal circadian rhythms, atherosclerosis, obesity-related gastroesophageal reflux, obesity-hypoventilation syndrome (Pickwickian syndrome), hypertriglyceridemia, low HDL cholesteremia, cardiovascular disease (for example, such as coronary artery heart disease (CHD), cerebrovascular disease, stroke, peripheral vascular disease, and sudden death), pain, osteoporosis-related disease, lower back pain, and anesthetic hypersensitivity. A compound of the present invention can be orally or parenterally administered, and can be prepared into a suitable administration form and used as a pharmaceutical composition for the prevention or treatment of diseases such as above.

In clinical use of a compound of the present invention, the compound may generally be administered after being prepared into various dosage forms with a pharmaceutically acceptable carrier in a manner suitable for the administration form. In this case, a variety of carriers commonly used in the field of pharmaceuticals can be used. Specific examples include gelatin, lactose, sucrose, titanium oxide, starch, microcrystalline cellulose, hydroxypropylmethyl cellulose, carboxymethyl cellulose, corn starch, microcrystalline wax, white vaseline, magnesium aluminometasilicate, anhydrous calcium phosphate, citric acid, trisodium citrate, hydroxypropyl cellulose, sorbitol, sorbitan fatty acid ester, polysorbate, sucrose fatty acid ester, polyoxyethylene, hydrogenated castor oil, polyvinylpyrrolidone, magnesium stearate, light anhydrous silicic acid, talc, vegetable oil, benzyl alcohol, gum arabic, propylene glycol, polyalkylene glycol, cyclodextrin, and hydroxypropylcyclodextrin.

Examples of the dosage forms prepared with the carrier include: solid preparations such as a tablet, a capsule, a granule, a powder, and a suppository; and liquid preparations such as a syrup, an elixir, and an injection. These can be prepared according to methods commonly used in the field of pharmaceuticals. The liquid preparation may be prepared by being dissolved or suspended in water or other suitable media before use. Specifically, the injection may be prepared by being dissolved or suspended in physiological saline or glucose solution as required, and may further include buffer or preservative.

The preparation may contain a compound of the present invention in a proportion of 1 to 99.9 weight %, preferably 1 to 60 weight % based on its pharmaceutical composition. The preparation may further contain other therapeutically effective compounds.

Specifically, the present invention provides a pharmaceutical composition that contains a pharmaceutically acceptable carrier, and a therapeutically effective amount of a compound of the present invention or a pharmaceutically acceptable salt thereof.

The therapeutically effective amount, as that term is used herein, means the amount of medicament that induces biological or medical events in tissues, systems, animals, or humans, as determined by researchers, veterinarians, physicians or other clinicians.

Specifically, in using a compound of the present invention for the prevention or treatment of the diseases such as above, the dose and dosing frequency can vary depending on such factors as the sex, age, and body weight of a patient, the level of symptoms, and the type and range of intended effect. Generally, in oral administration, the dosage generally may be 0.001 to 50 mg per kilogram body weight per day, given as a single dose or in multiple portions. Preferably, the dosage is about 0.01 to about 25 mg/kg per day, more preferably about 0.05 to about 10 mg/kg per day.

A compound of the present invention can be used for combination therapy with drugs (hereinafter, “co-drugs”) that are effective for diseases such as hypertension, obesity-related hypertension, hypertension-related disease, cardiac hypertrophy, left ventricular hypertrophy, metabolic disease, obesity, and obesity-related disease. The drug can be simultaneously, separately, or successively administered for the prevention or treatment of the disease. When using a compound of the present invention simultaneously with one or more co-drugs, they may be prepared as a pharmaceutical composition of a single administration form. However, in combination therapy, the co-drug and a composition containing a compound of the present invention may be simultaneously, separately, or successively administered to a subject in different packages. The packages may be given with a time lag.

The dose of the co-drug may be in accordance with that used in the clinic, and may be appropriately selected according to such factors as the subject, administration route, disease, and combination. The form of administration of the co-drug is not particularly limited, as long as the co-drug has been combined with a compound of the present invention at the time of administration.

Examples of the administration form include: (1) administration of a single preparation simultaneously prepared from a compound of the present invention and the co-drug, (2) simultaneous administration of two preparations separately prepared from a compound of the present invention and the co-drug, via the same administration route, (3) separate administration of two preparations prepared from a compound of the present invention and the co-drug, via the same administration route, (4) simultaneous administration of two preparations separately prepared from a compound of the present invention and the co-drug, via different administration routes, and (5) time-lagged administration of two preparations separately prepared from a compound of the present invention and the co-drug, via different administration routes (for example, the administration of a compound of the present invention and the co-drug in this order, and vice versa). The proportions of a compound of the present invention and the co-drug can be appropriately selected according to such factors as the subject, administration route, and disease.

Examples of the co-drug usable in the present invention include therapeutic drugs for diabetes mellitus, hyperlipidemia, and hypertension, and anti-obesity drugs. Two or more kinds of co-drugs may be used in combination in appropriate proportions.

The usefulness of a compound according to the present invention as a medicament is demonstrated by, for example, the following pharmacological test example 1.

### PHARMACOLOGICAL TEST EXAMPLE 1

### QRFP43 Binding Inhibition Test

The cDNA sequence [Accession No. NM_{—}198179] that codes for human QRFP receptor (GPR103) was cloned into an expression vector pEF1V5-HisB (Invitrogen). The expression vector so prepared was transfected into NFAT [β-Lactamase CHO-K1 host cells (Aurora) to obtain QRFP receptor (GPR103) expressing cells, using the cationic lipid method [see Proceedings of the National Academy of Sciences of the United States of America, Vol. 84, p. 7413 (1987)].

The membrane specimen prepared from the QRFP receptor (GPR103) expressing cells was incubated at 25° C. for 1 hour with a test compound and 20,000 cpm [¹²⁵I] QRFP43 (PerkinElmer, Inc.) in an assay buffer (50 mM Tris-HCl, 1 mM EDTA, and 0.1% BSA, pH 7.4), followed by filtration using a glass filter GF/C. After washing with a 50 mM Tris-HCl (containing 2 mM EDTA, 10 mM MgCl₂, and 0.04% Tween-20) buffer at pH 7.4, the radioactivity on the glass filter was determined using a gamma counter. Non-specific binding was measured in the presence of 1 μM peptide QRFP43, and 50% inhibition concentration (IC₅₀ value) of the test compound for the specific [¹²⁵I] QRFP43 binding was determined [see Endocrinology, Vol. 131, p. 2090 (1992)]. The results are shown in the table below.

TABLE 1

| Example | Structure | IC50(nM) |
|---|---|---|
| 2 | 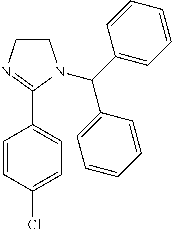 | 17 |
| 5 | 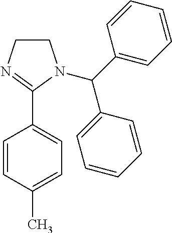 | 37 |
| 6 | 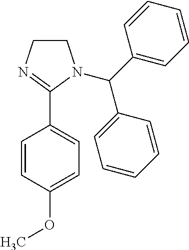 | 11 |

As can be seen from the results, the compounds of the present invention strongly inhibited the [¹²⁵I] QRFP43 binding to the QRFP43 receptor (GPR103).

As demonstrated above, a compound according to the present invention is expected to be useful for the prevention or treatment of a variety of diseases involving QRFP43 or 26RFa, including, for example, cardiovascular disease such as hypertension, arterial sclerosis, renal disease, heart disease, and angiospasm; bulimia; and metabolic disease such as obesity, diabetes mellitus, abnormal hormone secretion, hypercholesteremia, hyperlipidemia, gout, and fatty liver. A compound of the present invention can therefore be provided as a preventive or remedy for, for example, pain, abnormal circadian rhythms, atherosclerosis, obesity-related gastroesophageal reflux, obesity-hypoventilation syndrome (Pickwickian syndrome), hypertriglyceridemia, low HDL cholesteremia, cardiovascular disease (for example, such as coronary artery heart disease (CHD), cerebrovascular disease, stroke, peripheral vascular disease, and sudden death), pain, osteoporosis-related disease, lower back pain, and anesthetic hypersensitivity, and particularly for obesity.

### EXAMPLES

The present invention is described below more specifically based on examples. It should be noted, however, that the invention is in no way limited by the description of the following examples. As the column silica gel, Wakogel™ C-200 (Wako Pure Chemical Industries, Ltd.) was used. As the packed silica gel column, FLASH+™ cartridge, KP-Sil or FPNH, FLASH 12+M, FLASH 25+S, FLASH 25+M, FLASH 40+M, or the like (Biotage Japan), TC-C18 (Agilent), or Extend-C18 (Zorbax) was used. For preparative thin-layer chromatography, Kieselgel 60F254 (Merck) was used. Mass spectra were measured using Quattro II (Micromass). For the ¹HNMR measurement, JNM-AL400 (JEOL) or MERCURY vx400 (VARIAN), and ^{UNITY}INOVA 400 (VARIAN) were used. ZQ 2000 (Waters) was used for the mass spectral measurement. As the microwave synthesizer, INITIATOR (Biotage) was used.

### Example 1

### 1-(Diphenylmethyl)-2-phenyl-4,5-dihydro-1H-imidazole

Sodium hydride (60% oil, 216 mg) was added to a DMF solution (5 mL) of 2-phenyl-2-imidazoline (439 mg) under ice-cooled conditions, and the mixture was stirred at room temperature for 30 minutes. The mixture was further stirred for 16 hours after addition of bromodiphenylmethane (816 mg), and extracted with ethyl acetate after addition of a saturated sodium chloride aqueous solution. The organic layer was washed with saturated sodium bicarbonate solution and saturated brine, and concentrated after being dried over anhydrous sodium sulfate. The resulting residue was purified by silica gel column chromatography (methanol:chloroform=1:9) to give the title compound (650 mg) as a white solid.

1H-NMR (400 MHz, CDCl₃, δ ppm): 3.36 (2H, t, J=10.0 Hz), 3.84 (2H, t, J=10.0 Hz), 6.05 (1H, s), 7.14 (4H, d, J=6.8 Hz), 7.27-7.45 (9H, m), 7.55 (2H, d, J=8.4 Hz).

ESI-MS Found: m/z 313[M+H]⁺

### Example 2

### 2-(4-Chlorophenyl)-1-(diphenylmethyl)-4,5-dihydro-1H-imidazole

Ethylenediamine (33 mg) was added to a tert-butanol solution (5 mL) of 4-chlorobenzaldehyde (70 mg), and the mixture was stirred at room temperature for 30 minutes. The mixture was further stirred at 70° C. for 3 hours after addition of potassium carbonate (207 mg) and iodine (159 mg), and saturated sodium sulfite solution was added. After extracting the reaction mixture with chloroform, the organic layer was washed with saturated sodium bicarbonate solution and saturated brine, and concentrated after being dried over anhydrous sodium sulfate to give 2-(4-chlorophenyl)-2-imidazoline (96 mg) as a yellow solid. The solid so obtained was dissolved in a DMF solution (2 mL), which was then stirred at room temperature for 30 minutes after adding sodium hydride (60% oil, 30 mg) under ice-cooled conditions. The mixture was further stirred for 16 hours after addition of bromodiphenylmethane (136 mg), and extracted with ethyl acetate after addition of a saturated sodium chloride aqueous solution. The organic layer was washed with saturated sodium bicarbonate solution and saturated brine, and concentrated after being dried over anhydrous sodium sulfate. The resulting residue was purified by reverse-phase HPLC (0.1% formic acid-acetonitrile-H₂O) to give the title compound (37 mg) as a white solid.

1H-NMR (400 MHz, DMSO-d₆, δ ppm): 3.26 (2H, t, J=9.6 Hz), 3.68 (2H, t, J=9.6 Hz), 5.92 (1H, s), 7.08 (4H, d, J=7.2 Hz), 7.29 (2H, t, J=7.2 Hz), 7.36 (2H, t, J=7.2 Hz), 7.47 (2H, d, J=8.8 Hz), 7.52 (2H, d, J=8.8 Hz.

ESI-MS Found: m/z 347[M+H]⁺

### Example 3

### 2-(2-Chlorophenyl)-1-(diphenylmethyl)-4,5-dihydro-1H-imidazole

The title compound was obtained according to Example 2, using 2-chlorobenzaldehyde.

1H-NMR (400 MHz, DMSO-d₆, δ ppm): 3.27 (2H, t, J=10.0 Hz), 3.78 (2H, t, J=10.0 Hz), 5.48 (1H, s), 7.03-7.07 (4H, m), 7.29-7.34 (8H, m), 7.43 (1H, td, J=8.0, 0.8 Hz), 7.51 (1H, d, J=8.0 Hz).

ESI-MS Found: m/z 347[M+H]⁺

### Example 4

### 2-(3-Chlorophenyl)-1-(diphenylmethyl)-4,5-dihydro-1H-imidazole

The title compound was obtained according to Example 2, using 3-chlorobenzaldehyde.

1H-NMR (400 MHz, DMSO-d₆, δ ppm): 3.26 (2H, t, J=10.4 Hz), 3.68 (2H, t, J=10.4 Hz), 5.89 (1H, s), 7.06 (4H, d, J=7.2 Hz), 7.23-7.39 (7H, m), 7.43-7.47 (2H, m), 7.51-7.53 (1H, m).

ESI-MS Found: m/z 347[M+H]⁺

### Example 5

### 1-(Diphenylmethyl)-2-(4-methylphenyl)-4,5-dihydro-1H-imidazole

The title compound was obtained according to Example 2, using 4-methylbenzaldehyde.

1H-NMR (400 MHz, DMSO-d₆, δ ppm): 2.30 (3H, s), 3.27 (2H, t, J=10.0 Hz), 3.67 (2H, t, J=10.0 Hz), 5.95 (1H, s), 7.06 (4H, d, J=7.2 Hz), 7.23-7.36 (10H, m).

ESI-MS Found: m/z 327[M+H]⁺

### Example 6

### 1-(Diphenylmethyl)-2-(4-methoxyphenyl)-4,5-dihydro-1H-imidazole

The title compound was obtained according to Example 2, using 4-methoxybenzaldehyde.

1H-NMR (400 MHz, DMSO-d₆, δ ppm): 3.24 (2H, t, J=10.0 Hz), 3.62 (2H, t, J=10.0 Hz), 3.74 (3H, s), 5.97 (1H, s), 6.98 (2H, d, J=9.2 Hz), 7.06 (4H, d, J=7.6 Hz), 7.24-7.35 (6H, m), 7.40 (2H, d, J=8.8 Hz).

ESI-MS Found: m/z 343[M+H]⁺

### Example 7

### 1-(Diphenylmethyl)-2-[4-(trifluoromethyl)phenyl)-4,5-dihydro-1H-imidazole

The title compound was obtained according to Example 2, using 4-(trifluoromethyl)benzaldehyde.

1H-NMR (400 MHz, DMSO-d₆, δ ppm): 3.26 (2H, t, J=10.0 Hz), 3.69 (2H, t, J=10.0 Hz), 5.88 (1H, s), 7.06 (4H, d, J=7.6 Hz), 7.25-7.35 (6H, m), 7.65 (2H, d, J=8.0 Hz), 7.79 (2H, d, J=8.0 Hz).

ESI-MS Found: m/z 381[M+H]⁺

### Example 8

### 1-(Diphenylmethyl)-2-(4-methoxyphenyl)-4,4-dimethyl-4,5-dihydro-1H-imidazole

1,2-Diamino-2-methylpropane (264 mg) was added to a tert-butanol solution (10 mL) of 4-methoxybenzaldehyde (272 mg), and the mixture was stirred at room temperature for 30 minutes. Then, potassium carbonate (829 mg) and iodine (635 mg) were added to effect reaction at 140° C. for 7 minutes using a microwave synthesizer. The reaction mixture was allowed to cool to room temperature, and extracted with chloroform after addition of saturated sodium sulfite solution. The organic layer was washed with saturated sodium bicarbonate solution and saturated brine, and concentrated after being dried over anhydrous sodium sulfate. The residue was dissolved in a DMF solution (8 mL), and potassium carbonate (829 mg) and chlorodiphenylmethane (608 mg) were added to effect reaction at 160° C. for 20 minutes using a microwave synthesizer. After cooling, water was added to the reaction mixture, which was then extracted with ethyl acetate. The organic layer was washed with saturated sodium bicarbonate solution and saturated brine, and concentrated after being dried over anhydrous sodium sulfate. The resulting residue was purified by silica gel column chromatography (methanol:chloroform=1:9) to give the title compound (242 mg) as a white solid.

1H-NMR (400 MHz, CDCl₃, δ ppm): 1.16 (6H, s), 3.15 (2H, s), 3.82 (3H, s), 6.08 (1H, s), 6.90 (2H, d, J=8.4 Hz), 7.12 (4H, d, J=7.2 Hz), 7.21-7.36 (6H, m), 7.52 (2H, d, J=8.4 Hz).

ESI-MS Found: m/z 371[M+H]⁺

### Example 9

### 2-(4-Chlorophenyl)-1-(diphenylmethyl)-4,4-dimethyl-4,5-dihydro-1H-imidazole

The title compound was obtained as in Example 8, using 4-chlorobenzaldehyde. 1H-NMR (400 MHz, CDCl₃, δ ppm): 1.16 (6H, s)3.10 (2H, s), 5.95 (1H, s), 7.10 (4H, d, J=8.4 Hz), 7.29-7.38 (6H, m), 7.52 (2H, d, J=8.4 Hz).

ESI-MS Found: m/z 375[M+H]⁺

### INDUSTRIAL APPLICABILITY

A compound according to the present invention is expected to be useful for the prevention or treatment of a variety of diseases involving QRFP43 or 26RFa, including, for example, cardiovascular disease such as hypertension, arterial sclerosis, renal disease, heart disease, and angiospasm; bulimia; and metabolic disease such as obesity, diabetes mellitus, abnormal hormone secretion, hypercholesteremia, hyperlipidemia, gout, and fatty liver. A compound of the present invention can therefore be provided as a preventive or remedy for, for example, pain, abnormal circadian rhythms, atherosclerosis, obesity-related gastroesophageal reflux, obesity-hypoventilation syndrome (Pickwickian syndrome), hypertriglyceridemia, low HDL cholesteremia, cardiovascular disease (for example, such as coronary artery heart disease (CHD), cerebrovascular disease, stroke, peripheral vascular disease, and sudden death), pain, osteoporosis-related disease, lower back pain, and anesthetic hypersensitivity.

1. A compound represented by Formula (I) or a pharmaceutically acceptable salt thereof 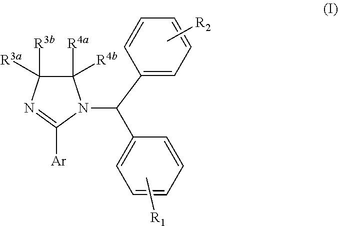
wherein R¹ and R² each independently represent a hydrogen atom, halogen, C₁₋₆alkyl, haloC₁₋₆alkyl, C₁₋₆alkyloxy, or haloC₁₋₆alkyloxy,
R^{3a}, R^{3b}, R^{4a} and R^{4b} each independently represent a hydrogen atom, C₁₋₆alkyl, or haloC₁₋₆alkyl, and
Ar represents aryl, wherein the aryl may be substituted with 1 to 3 substituents selected from a group consisting of halogen, cyano, C₁₋₆alkyl, haloC₁₋₆alkyl, C₁₋₆alkyloxy, haloC₁₋₆alkyloxy, hydroxy, amino, monoC₁₋₆alkylamino, diC₁₋₆alkylamino, C₁₋₆alkyloxycarbonyl, C₁₋₆alkylcarbonylamino, C₁₋₆alkylcarbonyl(C₁₋₆alkyl)amino, C₁₋₆alkyloxycarbonylamino, C₁₋₆alkyloxycarbonyl(C₁₋₆alkyl)amino, C₁₋₆alkylsulfanyl, C₁₋₆alkylcarbonyl, and C₁₋₆alkylcarbonyloxy.

2. The compound or the pharmaceutically acceptable salt thereof according to claim 1, wherein R¹ and R² are each independently a hydrogen atom, halogen, or C₁₋₆alkyl.

3. The compound or the pharmaceutically acceptable salt thereof according to claim 1, wherein R^{3a}, R^{3b}, R^{4a} and R^{4b} are each independently a hydrogen atom or C₁₋₆alkyl.

4. The compound or the pharmaceutically acceptable salt thereof according to claim 1, wherein the aryl for Ar is a phenyl or naphthyl.

5. The compound or the pharmaceutically acceptable salt thereof according to claim 1, wherein the aryl for Ar is a phenyl.

6. The compound or the pharmaceutically acceptable salt thereof according to claim 1, wherein the aryl for Ar is a naphthyl.

7. The compound or the pharmaceutically acceptable salt thereof according to claim 1, wherein the optional substituent of Ar is selected from a group consisting of halogen, cyano, C₁₋₆alkyl, haloC₁₋₆alkyl, C₁₋₆alkyloxy, haloC₁₋₆alkyloxy, C₁₋₆alkylcarbonylamino, and C₁₋₆alkylsulfanyl.

8. The compound or the pharmaceutically acceptable salt thereof according to claim 1, wherein Ar is selected from a group consisting of phenyl, 4-chlorophenyl, 4-methylphenyl, 4-methoxyphenyl, 4-ethoxyphenyl, 4-cyanophenyl and 4-methylthiophenyl.

9. The compound or the pharmaceutically acceptable salt thereof according to claim 1, wherein R¹ and R² are each independently a hydrogen atom, halogen, or C₁₋₆alkyl, R^{3a}, R^{3b}, R^{4a} and R^{4b} are each independently a hydrogen atom or C₁₋₆alkyl, and the aryl for Ar is a phenyl or naphthyl.

10. The compound or the pharmaceutically acceptable salt thereof according to claim 1, wherein R¹ and R² are each independently a hydrogen atom, halogen, or C₁₋₆alkyl, R^{3a}, R^{3b}, R^{4a} and R^{4b} are each independently a hydrogen atom or C₁₋₆alkyl, and Ar is a phenyl or naphthyl, wherein the phenyl or the naphthyl may be substituted with a substituent selected from a group consisting of halogen, cyano, C₁₋₆alkyl, haloC₁₋₆alkyl, C₁₋₆alkyloxy, haloC₁₋₆alkyloxy, C₁₋₆alkylcarbonylamino and C₁₋₆alkylsulfanyl.

11. The compound or the pharmaceutically acceptable salt thereof according to claim 1, wherein R¹ and R² are each independently a hydrogen atom, halogen, or C₁₋₆alkyl, R^{3a}, R^{3b}, R^{4a} and R^{4b} are each independently a hydrogen atom or C₁₋₆alkyl, and Ar is one of phenyl, 4-chlorophenyl, 4-methylphenyl, 4-methoxyphenyl, 4-ethoxyphenyl, 4-cyanophenyl, and 4-methylthiophenyl.

12. The compound or the pharmaceutically acceptable salt thereof according to claim 1, wherein the compound of the Formula (I) is selected from a group consisting of:
1-(diphenylmethyl)-2-phenyl-4,5-dihydro-1H-imidazole,
2-(4-chlorophenyl)-1-(diphenylmethyl)-4,5-dihydro-1H-imidazole,
2-(2-chlorophenyl)-1-(diphenylmethyl)-4,5-dihydro-1H-imidazole,
2-(3-chlorophenyl)-1-(diphenylmethyl)-4,5-dihydro-1H-imidazole,
1-(diphenylmethyl)-2-(4-methylphenyl)-4,5-dihydro-1H-imidazole,
1-(diphenylmethyl)-2-(4-methoxyphenyl)-4,5-dihydro-1H-imidazole,
1-(diphenylmethyl)-2-[4-(trifluoromethyl)phenyl)-4,5-dihydro-1H-imidazole,
1-(diphenylmethyl)-2-(4-methoxyphenyl)-4,4-dimethyl-4,5-dihydro-1H-imidazole, and
2-(4-chlorophenyl)-1-(diphenylmethyl)-4,4-dimethyl-4,5-dihydro-1H-imidazole.

13. A pharmaceutical composition, which comprises the compound of claim 1, and a pharmaceutically acceptable carrier.

14. (canceled)

15. A method for treating obesity in a subject in need thereof which comprises administering to the subject a therapeutically effective amount of a compound of claim 1, or a pharmaceutically acceptable salt thereof.
